(19) United States
(12) Patent Application Publication
(10) Pub. No.: US 2020/0320698 A1
(43) Pub. Date: Oct. 8, 2020

(54) SPECIMEN-SAMPLE ESTIMATION APPARATUS, SPECIMEN-SAMPLE ESTIMATION METHOD, AND COMPUTER-READABLE RECORDING MEDIUM

(71) Applicant: Olympus Corporation, okyo (JP)

(72) Inventor: Ken IOKA, Tokyo (JP)

(73) Assignee: Olympus Corporation, Tokyo (JP)

(21) Appl. No.: 16/867,648

(22) Filed: May 6, 2020

Related U.S. Application Data
(63) Continuation of application No. PCT/JP2017/043888, filed on Dec. 6, 2017.

(51) Int. Cl. G06T 7/00; H04N 5/225; G06T 7/62; G02B 21/36; G02B 21/06; G02B 26/00; G01N 21/27; G01N 33/483; A61B 1/05; A61B 1/06; A61B 10/04; A61B 1/00

(52) U.S. Cl. CPC G06T 7/0012; A61B 2010/045; G06T 7/62; G02B 21/365; G02B 21/06; G02B 26/007; H04N 5/2254; G01N 21/27; G01N 33/4833; A61B 1/05; A61B 1/0646; A61B 10/04; A61B 1/00133; G06T 2207/10056; G06T 2207/30024; G06T 2207/30204; G01N 2201/063; H04N 5/2253

(57) ABSTRACT

A specimen-sample estimation apparatus includes: a light emitter configured to emit white light to irradiate light to a specimen sample including a living tissue; special light filters that are insertable and removable to and from an optical path of the white light, each special light filter being configured to transmit light having a different wavelength band; a driver configured to move one of the special light filters to the optical path; an imager configured to image the specimen sample to generate image data; and a processor including hardware, the processor being configured to detect a core tissue region of the living tissue appearing in an image corresponding to the image data, calculate an amount of tissue of the living tissue, based on the detected core tissue region, and determine whether the calculated amount of tissue is equal to or larger than a predetermined threshold.

### CROSS-REFERENCE TO RELATED APPLICATION

This application is a continuation of International Application No. PCT/JP2017/043888, filed on Dec. 6, 2017, the entire contents of which are incorporated herein by reference.

### BACKGROUND

### 1. Technical Field

The present disclosure relates to a specimen-sample estimation apparatus, a specimen-sample estimation method, and a computer-readable recording medium that estimate specimen samples including a living tissue collected by endoscopic needle biopsy.

### 2. Related Art

In the related art, in intraoperative rapid cytodiagnosis in endoscopic needle biopsy, it is necessary to reduce the number of biopsy sample collection, and to provide a necessary and sufficient amount of living tissue for pathological diagnosis to obtain definitive diagnosis. For this, a technique has been known in which an illumination light of a predetermined wavelength band is irradiated to a specimen sample tissue including a living tissue and blood collected by a biopsy needle, and it is determined whether a specimen cell is present in the sample tissue of the subject based on a transmission image formed by transmission light that is the illumination light that has passed therethrough (refer to Japanese Patent No. 5861225). In this technique, by irradiating light, a wavelength of the maximum light emission intensity of which is 600 nanometers (nm) to 610 nm is irradiated to a specimen sample tissue. The light is absorbed by blood included in the specimen sample tissue, and it thus becomes possible to discriminate between a living tissue and blood from the transmission image.

### SUMMARY

In some embodiments, a specimen-sample estimation apparatus includes: a light emitter configured to emit white light to irradiate light to a specimen sample including a living tissue; a plurality of special light filters that are insertable and removable to and from an optical path of the white light emitted by the light emitter, each special light filter being configured to transmit light having a different wavelength band; a driver configured to move one of the special light filters to the optical path; an imager configured to image the specimen sample to which the light has been irradiated by the light emitter, to generate image data; and a processor including hardware, the processor being configured to detect a core tissue region of the living tissue appearing in an image corresponding to the image data generated by the imager, calculate an amount of tissue of the living tissue, based on the detected core tissue region, and determine whether the calculated amount of tissue is equal to or larger than a predetermined threshold.

In some embodiments, a specimen-sample estimation method including: moving one of a plurality of special light filters to an optical path of white light, each special light filter being configured to transmit light having a different wavelength band; irradiating the white light to a specimen sample including a living tissue; imaging the specimen sample to which the white light has been irradiated, to generate image data; detecting a core tissue region of the living tissue appearing in an image corresponding to the image data; calculating an amount of tissue of the living tissue based on the core tissue region; and determining whether the amount of tissue is equal to or larger than a predetermined threshold.

In some embodiments, provided is a non-transitory computer-readable recording medium with an executable program stored thereon. The program causes a specimen-sample estimation apparatus to execute: moving one of a plurality of special light filters to an optical path of white light, each special light filter being configured to transmit light having a different wavelength band; irradiating the white light to a specimen sample including a living tissue; imaging the specimen sample to which the white light has been irradiated, to generate image data; detecting a core tissue region of the living tissue appearing in an image corresponding to the image data; calculating an amount of tissue of the living tissue based on the core tissue region; and determining whether the amount of tissue is equal to or larger than a predetermined threshold.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a schematic diagram illustrating a schematic configuration of a specimen-sample estimation apparatus according to a first embodiment;

FIG. 2 is a flowchart illustrating an outline of processing performed by the specimen-sample estimation apparatus according to the first embodiment;

FIG. 3 is a schematic diagram illustrating a schematic configuration of a specimen-sample estimation apparatus according to a second embodiment;

FIG. 4 is a schematic illustration of spectral absorptance of respective tissues included in a living tissue;

FIG. 5 is a flowchart illustrating an outline of processing performed by the specimen-sample estimation apparatus according to the second embodiment of the present disclosure;

FIG. 6 is a schematic illustration of spectral absorptance of respective tissues included in a living tissue;

FIG. 7 is a schematic diagram illustrating a schematic configuration of a specimen-sample estimation apparatus according to a third embodiment;

FIG. 8 is a schematic diagram illustrating a schematic configuration of a specimen-sample estimation apparatus according to a fourth embodiment;

FIG. 9 is a flowchart illustrating an outline of processing performed by the specimen-sample estimation apparatus according to the fourth embodiment;

FIG. 10 illustrates an example of an image when a separating unit according to the fourth embodiment separates a blood region and a tissue specimen region;

FIG. 11 is a schematic diagram illustrating a schematic configuration of a specimen-sample estimation apparatus according to a fifth embodiment;

FIG. 12 is a flowchart illustrating an outline of processing performed by the specimen-sample estimation apparatus according to the fifth embodiment; and

FIG. 13 illustrates an example of an image in which a detecting unit according to the fifth embodiment has detected a core tissue region.

### DETAILED DESCRIPTION

In the following, forms (hereinafter, “embodiments”) to implement the disclosure will be described. In the embodiments, a specimen-sample estimation apparatus that estimates a specimen sample including a living tissue that is collected by biopsy will be described as an example. The disclosure is not limited to the following embodiments. Moreover, identical components will be explained with identical reference signs assigned thereto in the drawings.

### First Embodiment

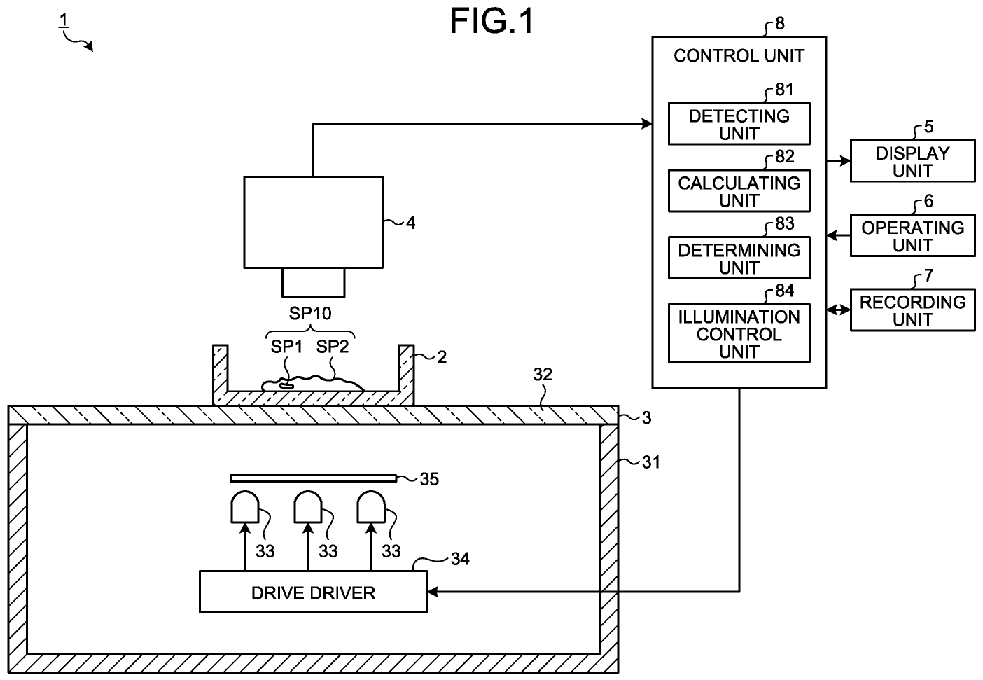

FIG. 1 is a schematic diagram illustrating a schematic configuration of a specimen-sample estimation apparatus according to a first embodiment. A specimen-sample estimation apparatus 1 illustrated in FIG. 1 includes an illuminating unit 3 on which a specimen sample container 2, such as a petri dish, containing a specimen sample SP10 including at least a living tissue SP1 and blood SP2 that have been collected by fine needle aspiration with a puncture needle, by forceps biopsy, or brush biopsy is mounted, and that irradiates light to the specimen sample SP10, an imaging unit 4 that images light passed through the specimen sample SP10 to generate image data, a display unit 5 that is capable of displaying an image corresponding to the image data generated by the imaging unit 4, an operating unit 6 that receives an input of an instruction signal to operate respective components of the specimen-sample estimation apparatus 1, a recording unit 7 that records various kinds of programs and data executed by the specimen-sample estimation apparatus 1, and image data, and a control unit 8 that controls the respective components constituting the specimen-sample estimation apparatus 1 in a centralized manner.

The illuminating unit 3 includes a case portion 31 that has a form of a casing, and that is formed with a light-shielding member, a lid portion 32 that is formed with a transparent member, such as glass, multiple light emitting units that irradiate light to the specimen sample SP10, a drive driver 34 that supplies electric power to the light emitting units 33 under control of the control unit 8, and a plate-shaped filter 35 that converts light emitted by the light emitting unit 33 into scattered light. The light emitting unit 33 emits white light. The white light emitted by the light emitting unit 33 passes through the filter 35, the lid portion 32, and the container 2 for examination, to be irradiated to the specimen sample SP10. The light emitting unit 33 is composed of, for example, a white light emitting diode (LED). In the first embodiment, the illuminating unit 3 is structured with multiple pieces of the light emitting units 33, but the number thereof may be changed appropriately.

The imaging unit 4 includes an optical system constituted of, at least, a lens to form an image of a subject and the like, and an image sensor, such as a charge coupled device (CCD) and a complementary metal oxide semiconductor (CMOS). The imaging unit 4 generates image data by receiving light that has passed through the specimen sample SP10, and by subjecting the light to photoelectric conversion, and outputs the image data to the control unit 8.

The display unit 5 displays an image corresponding to the image data input from the control unit 8, and various kinds of information relating to the specimen-sample estimation apparatus 1 under control of the control unit 8. The display unit 5 includes a display panel of a liquid crystal, an organic electroluminescence (EL), or the like.

The operating unit 6 receives an input of an instruction signal to specify various kinds of operations of the specimen-sample estimation apparatus 1, and outputs the received instruction signal to the control unit 8. The operating unit 6 includes a mouse, a keyboard, a foot switch, a button, a jog dial, a touch panel, and the like.

The recording unit 7 is constituted of a synchronous dynamic random access memory (SDRAM), a flash memory, or the like. The recording unit 7 records image data, various kinds of programs that are executed by the specimen-sample estimation apparatus 1, and data being processed. The recording unit 7 may be constituted of an externally detachable recording medium, such as a memory card.

The control unit 8 is constituted of any one of a central processing unit (CPU), a field programmable gate array (FPGA), and an application specific integrated circuit (ASIC). The control unit 8 includes a detecting unit 81, a calculating unit 82, a determining unit 83, and an illumination control unit 84.

The detecting unit 81 detects a core tissue region of a living tissue that appears in an image corresponding to image data generated by the imaging unit 4. Specifically, the detecting unit 81 detects a core tissue region of a living tissue that appears in an image corresponding to image data generated by the imaging unit 4 based on an instruction signal that specifies a position or a region of the living tissue appearing in the image input from the operating unit 6. For example, an example of a core tissue region corresponds to a part of a piece of living body in a specimen sample including blood and a piece of tissue that have been collected by biopsy.

The calculating unit 82 calculates an amount of tissue of a living body tissue based on the core tissue region detected by the detecting unit 81. Specifically, the calculating unit 82 calculates at least one of a length, a width, an area, and a volume of the core tissue region, as the amount of tissue.

The determining unit 83 determines whether the amount of tissue calculated by the calculating unit 82 is equal to or larger than a predetermined threshold.

The illumination control unit 84 controls drive of the illumination unit 3. Specifically, the illumination control unit 84 performs a control to cause the illuminating unit 3 to irradiate white light to the specimen sample SP10 contained in the container 2 for examination according to an instruction signal input from the operating unit 6.

Processing of Specimen-Sample Estimation Apparatus

Next, processing performed by the specimen-sample estimation apparatus 1 will be described.

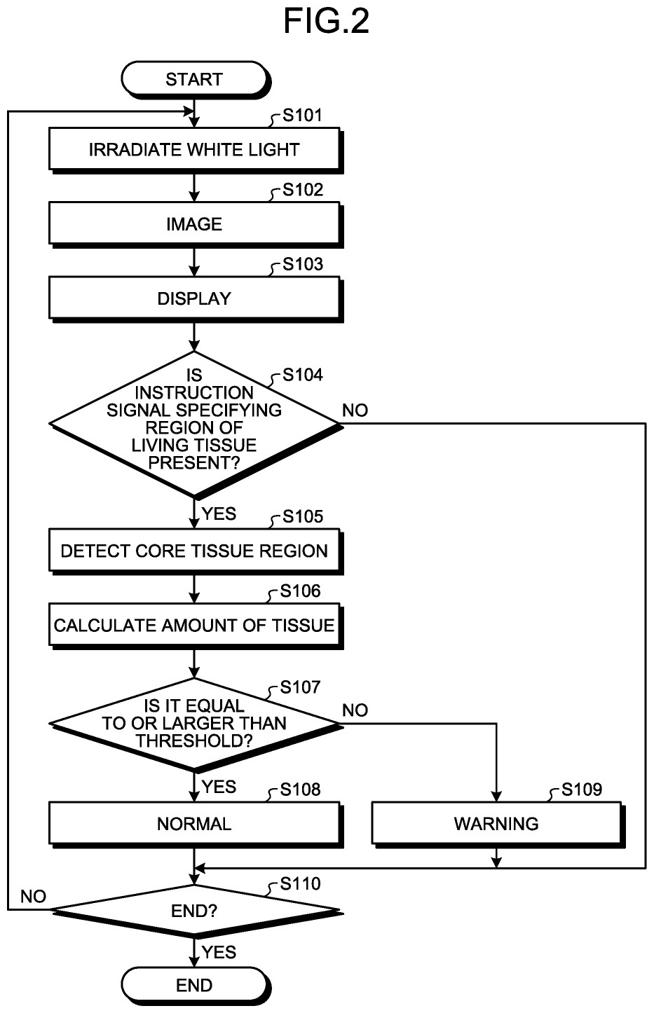

FIG. 2 is a flowchart illustrating an outline of the processing performed by the specimen-sample estimation apparatus 1.

As illustrated in FIG. 2, first, the illumination control unit 84 causes the illumination unit 3 to irradiate white light to the specimen sample SP10 contained in the container 2 for examination (step S101).

Subsequently, the imaging unit 4 images the specimen sample SP10 contained in the container 2 for examination to generate image data (step S102).

Thereafter, the display unit 5 displays an image corresponding to the image data generated by the imaging unit 4 (step S103). Thus, an examination operator can view the image of the specimen sample SP10 collected by puncture of biopsy through an endoscope or an ultrasound probe.

Subsequently, when an instruction signal specifying a region of a living tissue that appears in the image input by the operating unit 6 is input (step S104: YES), the detecting unit 81 detects a core tissue region of the living tissue appearing in the image corresponding to the image data generated by the imaging unit 4 based on the instruction signal specifying a position or a region of the living tissue appearing in the image input by the operating unit 6 (step S105).

Thereafter, the calculating unit 82 calculates an amount of tissue of the living tissue based on the core tissue region detected by the detecting unit 81 (step S106). Specifically, the calculating unit 82 calculates a width Oh and a height Ov of the living tissue by following Equation (1) when a focal length of the imaging unit 4 is F, a distance from the imaging unit 4 to the specimen sample SP10 is D, a pixel size of an imaging sensor of the imaging unit 4 in a horizontal direction is H, a pixel size of the imaging sensor of the imaging unit 4 in a vertical direction is V, the width of the living tissue is Oh, and the height of the living tissue is Ov.

D/F=Oh/H=Ov/V (1)

That is, the expansion of Equation (1) above is

Oh=(D×H)/F (2)

Ov=(D×V)/F (3)

The calculating unit 82 then calculates an area of the living tissue (Oh×Ov) from Equation (2) and Equation (3). The calculating unit 82 acquires respective values of the focal length F, the distance D, the pixel size H, the pixel size V from EXIF when the imaging unit 4 generates the image data. Moreover, other than the calculation method using above (1) to (3), the calculating unit 82 may calculate a volume as the amount of tissue based on a diameter of a puncture needle used in treatment. In this case, the examination operator, such as a doctor, can input a diameter of the puncture needle from the operating unit 6. Of course, the examination operator, such as a doctor, may input a diameter of the puncture needle and a length in a puncture direction (insertion distance) from the operating unit 6. Furthermore, the calculating unit 82 calculates an area of the living tissue but, not limited thereto, the calculating unit 82 may calculate a length or a width of the core tissue region. Moreover, the calculating unit 82 may calculate the amount of tissue of the living tissue based on a marker indicating dimensions engraved or printed on the specimen sample container 2 that appears in an image corresponding to image data.

Subsequently, the determining unit 83 determines whether the amount of tissue calculated by the calculating unit 82 is equal to or larger than a predetermined threshold (step S107). When the determining unit 83 determines that the amount of tissue calculated by the calculating unit 82 is equal to or larger than the threshold (step S107: YES), the specimen-sample estimation apparatus 1 shifts to step S108 described later. On the other hand, when the determining unit 83 determines that the amount of tissue calculated by the calculating unit 82 is not equal to or larger than the threshold (step S107: NO). The specimen-sample estimation apparatus 1 shifts to step S109 described later.

At step S108, the display unit 5 displays information indicating that the amount of tissue of the living tissue of the specimen sample SP10 is normal. Thus, the examination operator, such as a doctor, can understand intuitively that a sufficient amount of tissue of the living tissue of the specimen sample SP10 for biopsy has been obtained. After step S108, the specimen-sample estimation apparatus 1 shifts to step S110 described later.

At step S109, the display unit 5 displays a warning indicating that the amount of tissue of the living tissue of the specimen sample SP10 is not normal. Thus, the examination operator, such as a doctor, can understand intuitively that a sufficient amount of tissue of the living tissue of the specimen sample SP10 for biopsy cannot be obtained. After step S109, the specimen-sample estimation apparatus 1 shifts to step S110 described later.

Subsequently, when an instruction signal to instruct an end of evaluation of the specimen sample SP10 is input from the operating unit 6 (step S110: YES), the specimen-sample estimation apparatus 1 ends the processing. On the other hand, when the instruction signal to instruct an end of evaluation of the specimen sample SP10 is not input from the operating unit 6 (step S110: NO), the specimen-sample estimation apparatus 1 returns to step S101 described above.

At step S104, when an instruction signal specifying a region of the living tissue appearing in the image input by the operating unit 6 is not input (step S104: NO), the specimen-sample estimation apparatus 1 shifts to step S110.

According to the first embodiment described above, because the determining unit 83 determines whether an amount of tissue calculated by the calculating unit 82 is equal to or larger than the threshold, it is possible to estimate whether a sufficient amount of tissue of a living tissue necessary for pathological diagnosis has been obtained.

Moreover, according to the first embodiment, because the detecting unit 81 detects a core tissue region based on an instruction signal received by the operating unit 6, it is possible to detect a region of a living tissue reliably.

Furthermore, according to the first embodiment, because the display unit 5 displays a warning when the determining unit 83 determined that an amount of tissue calculated by the calculating unit 82 is not equal to or larger than a predetermined threshold, the examination operator, such as a doctor, can understand intuitively that a sufficient amount of tissue of the living tissue of the specimen sample SP10 for biopsy cannot be obtained.

In the first embodiment, the display unit 5 displays a warning when the determining unit 83 determines that an amount of tissue calculated by the calculating unit 82 is not equal to or larger than a predetermined threshold, but an output unit, such as a speaker, may output a warning, or the warning may be given by light.

### Second Embodiment

Next, a second embodiment will be described. In the second embodiment, while switched to either one, special light and white light are irradiated to a specimen sample. Hereinafter, a configuration of a specimen-sample estimation apparatus according to the second embodiment will be described, and then processing performed by the specimen-sample estimation apparatus according to the second embodiment will be described. Note that identical reference signs are assigned to components identical to those in the specimen-sample estimation apparatus 1 according to the first embodiment, and explanation thereof will be omitted.

Configuration of Specimen-Sample Estimation Apparatus

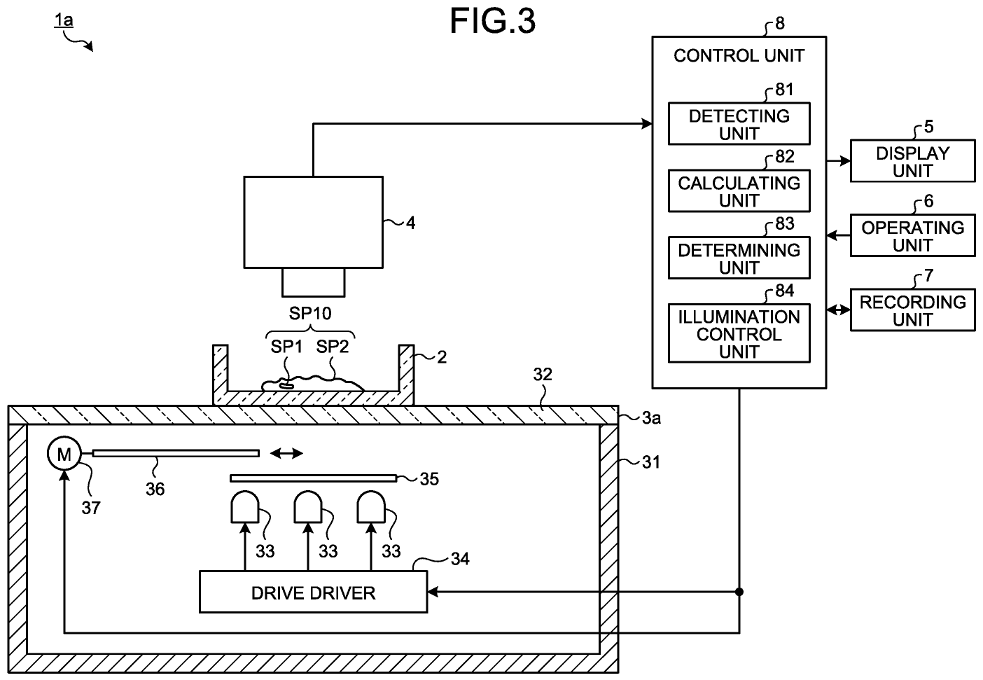

FIG. 3 is a schematic diagram illustrating a schematic configuration of a specimen-sample estimation apparatus according to a second embodiment. A specimen-sample estimation apparatus 1a illustrated in FIG. 3 includes an illuminating unit 3a in place of the illuminating unit 3 in the specimen-sample estimation apparatus 1 according to the first embodiment described above.

The illuminating unit 3a illuminates white light and special light to the specimen sample SP10 contained in the container 2 for examination, while switching therebetween, under control of the control unit 8. The illuminating unit 3a includes a special light filter 36 and a driving unit 37 in addition to the components of the illuminating unit 3 according to the first embodiment described above.

The special light filter 36 transmits light of a predetermined wavelength band. Specifically, the special light filter 36 transmits light of a wavelength band of 400 nm to 450 nm or 580 nm to 650 nm.

The driving unit 37 moves the special light filter 36 to an optical path of white light emitted by the light emitting unit 33 under control of the illumination control unit 84. The driving unit 37 is constituted of a motor, and the like.

Spectral Absorptance of Respective Tissues in Living Tissue

Next, spectral absorptances of respective tissues included in a living tissue will be explained.

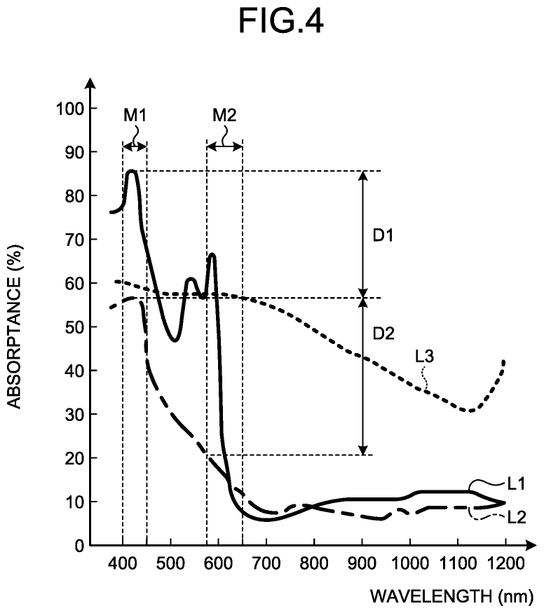

FIG. 4 is a schematic illustration of spectral absorptance of respective tissues included in a living tissue. In FIG. 4, a horizontal axis is for wavelength (nm), and a vertical axis is for absorptance (%). In FIG. 4, a curve L1 shows a spectral absorptance of hemoglobin, a curve L2 shows a spectral absorptance of collagen, and a curve L3 shows a spectral absorptance of melanin.

As the curve L1 and the curve L2 in FIG. 4 show, hemoglobin and collagen have different spectral absorptances. Furthermore, hemoglobin and collagen have large differences D1, D2 in spectral absorptance with light M1 of a wavelength band, 400 nm to 450 nm, and with light M2 of a wavelength band, 580 nm to 650 nm. Therefore, for the special light filter 36, the transmission property is set to transmit the light M1 of the wavelength band, 400 nm to 450 nm, or the light M2 of the wavelength band, 580 nm to 650 nm, and a half width is set to 30 nm or smaller. Thus, a narrowband light can be irradiated to the specimen sample SP10 as the special light. As a result, the contrast between a collagen tissue and blood included in the specimen sample SP10 can be increased, and it becomes possible to discriminate therebetween easily. In the second embodiment, it is supposed that the transmission property of the special light filter 36 is a wavelength band of 580 nm to 650 nm.

Processing of Specimen-Sample Estimation Apparatus

Next, processing performed by the specimen-sample estimation apparatus 1a will be described.

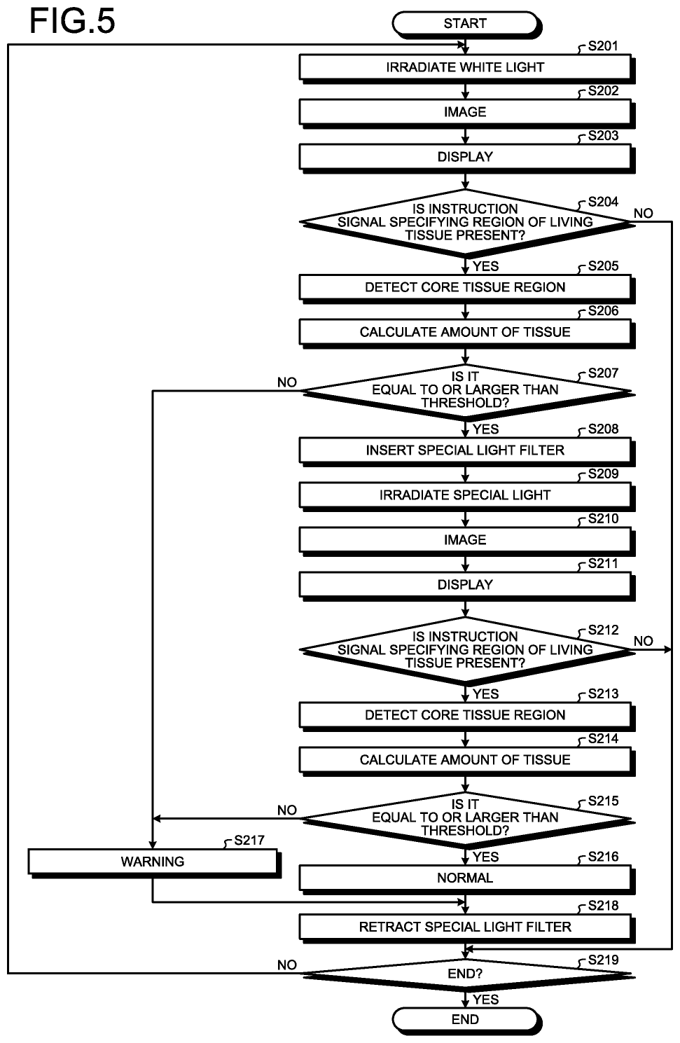

FIG. 5 is a flowchart illustrating an outline of the processing performed by the specimen-sample estimation apparatus 1a. In FIG. 5, step S201 to Step S206 correspond to step S101 to step S106 in FIG. 2 described above, respectively.

At step S207, the determining unit 83 determines whether an amount of tissue calculated by the calculating unit 82 is equal to or larger than a predetermined threshold (step S207). When the determining unit 83 determines that the amount of tissue calculated by the calculating unit 82 is equal to or larger than the predetermined threshold (step S207: YES), the specimen-sample estimation apparatus 1a shifts to step S208 described later. On the other hand, when the determining unit 83 determines that the amount of tissue calculated by the calculating unit 82 is not equal to or larger than the predetermined threshold (step S207: NO), the specimen-sample estimation apparatus 1a shifts to step S217 described later.

At step S208, the illumination control unit 84 drives the driving unit 37, and inserts the special light filter 36 in the optical path of the light emitting unit 33.

Subsequently, the illumination control unit 84 causes the illuminating unit 3a to irradiate special light to the specimen sample SP10 contained in the container 2 for examination (step S209).

Subsequently, the imaging unit 4 images the specimen sample SP10 to which the special light has been irradiated, to generate image data (step S210).

Thereafter, the display unit 5 displays an image corresponding to the image data generated by the imaging unit 4 (step S211). Thus, the examination operator, such as a doctor, can view an image in which the contrast between the specimen sample SP10, such as collagen, and the blood SP2 is increased.

Subsequently, when an instruction signal specifying a region of the living tissue appearing in the image input by the operating unit 6 is input (step S212: YES), the detecting unit 81 detects a core tissue region of the living tissue appearing in the image corresponding to the image data generated by the imaging unit 4 based on the instruction signal specifying a position or a region of the living tissue appearing in the image input from the operating unit 6 (step S213).

Thereafter, the calculating unit 82 calculates an amount of tissue of the living tissue based on the core tissue region detected by the detecting unit 81 (step S214).

Subsequently, the determining unit 83 determines whether the amount of tissue calculated by the calculating unit 82 is equal to or larger than a predetermined threshold (step S215). When the determining unit 83 determines that the amount of tissue calculated by the calculating unit 82 is equal to or larger than the predetermined threshold (step S215: YES), the specimen-sample estimation apparatus 1a shifts to step S216 described later. On the other hand, when the determining unit 83 determines that the amount of tissue calculated by the calculating unit 82 is not equal to or larger than the predetermined threshold (step S215: NO), the specimen-sample estimation apparatus 1a shifts to step S217 described later.

At step S216, the display unit 5 displays information indicating that the amount of tissue of the living tissue of the specimen sample SP10 is normal. Thus, the examination operator, such as a doctor, can understand intuitively that a sufficient amount of tissue of the living tissue of the specimen sample SP10 for biopsy has been obtained. After step S216, the specimen-sample estimation apparatus 1a shifts to step S218 described later.

At step S217, the display unit 5 displays a warning indicating that the amount of tissue of the living tissue of the specimen sample SP10 is not normal. Thus, the examination operator, such as a doctor, can understand intuitively that a sufficient amount of tissue of the living tissue of the specimen sample SP10 for biopsy cannot be obtained. After step S217, the specimen-sample estimation apparatus 1a shifts to step S218 described later.

At step S218, the illumination control unit 84 drives the driving unit 37, and retracts the special light filter 36 from the optical path of the light emitting unit 33.

Subsequently, when an instruction signal to instruct an end of evaluation of the specimen sample SP10 is input from the operating unit 6 (step S219: YES), the specimen-sample estimation apparatus 1a ends the processing. On the other hand, when the instruction signal to instruct an end of evaluation of the specimen sample SP10 is not input from the operating unit 6 (step S219: NO), the specimen-sample estimation apparatus 1a returns to step S201 described above.

At step S212, when an instruction signal specifying a region of the living tissue appearing in the image input by the operating unit 6 is not input (step S212: NO), the specimen-sample estimation apparatus 1a shifts to step S219.

According to the second embodiment described above, because the contrast between the living tissue SP1 and the blood SP2 can be increased by irradiating special light to the specimen sample SP10 by the illuminating unit 3a, it is possible to estimate whether a sufficient amount of tissue of a living tissue necessary for pathological diagnosis has been obtained.

### Modification of Second Embodiment

In the first embodiment described above, the transmission property of the special light filter 36 is set based on characteristics of spectral absorptance of collagen and hemoglobin, as types of the living tissues. However, not limited thereto, for example, fat and hemoglobin may be applied as the type of the living tissue. In the following, spectral absorptance of fat and hemoglobin as types of the living tissue will be described.

Spectral Absorptance of Respective Tissues in Living Tissue

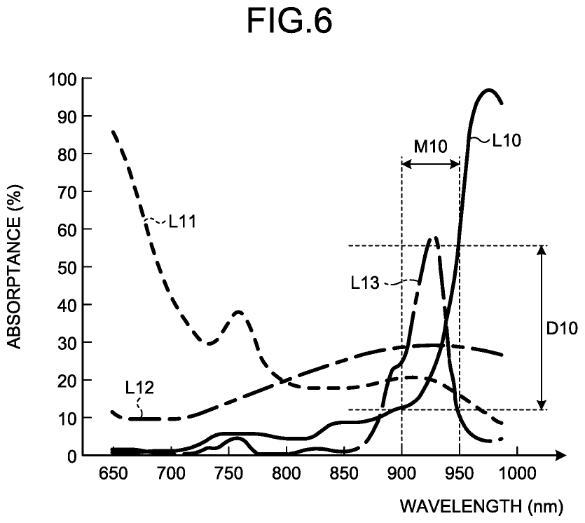

FIG. 6 is a schematic illustration of spectral absorptance of respective tissues included in a living tissue. In FIG. 6, a horizontal axis is for wavelength (nm), and a vertical axis is for absorptance (%). Moreover, in FIG. 6, a curve L10 shows a spectral absorptance of water, a curve L11 shows a spectral absorptance of deoxygenated hemoglobin, a curve L12 shows a spectral absorptance of oxygenated hemoglobin, and a curve L13 shows a spectral absorptance of fat.

As the curve L11, the curve L12, and the curve L13 in FIG. 6 show, deoxygenated hemoglobin, oxygenated hemoglobin, and fat have different spectral absorptances. Furthermore, a difference in spectral absorptance among deoxygenated hemoglobin, oxygenated hemoglobin, and fat becomes largest with light M10 of a wavelength band, 900 nm to 950 nm, which is a near infrared spectrum. Therefore, for the special light filter 36, the transmission property is set to transmit the light M10 of the wavelength band, 900 nm to 950 nm, and a half width is set to 30 nm or smaller. Thus, a narrowband light can be irradiated to the specimen sample SP10 as the special light. As a result, the contrast between a fat tissue and blood included in the specimen sample SP10 can be increased, and it becomes possible to discriminate therebetween easily.

According to the modification of the second embodiment described above, effects similar to those of the second embodiment described above can be produced.

### Third Embodiment

Next, a third embodiment will be described. In the third embodiment, the wavelength band of the special light to be irradiated to a specimen sample is changed according to a type of the specimen sample. Hereinafter, a configuration of a specimen-sample estimation apparatus according to the third embodiment will be described. Note that identical reference signs are assigned to components identical to those in the specimen-sample estimation apparatus 1a according to the second embodiment, and explanation thereof will be omitted.

Configuration of Specimen-Sample Estimation Apparatus

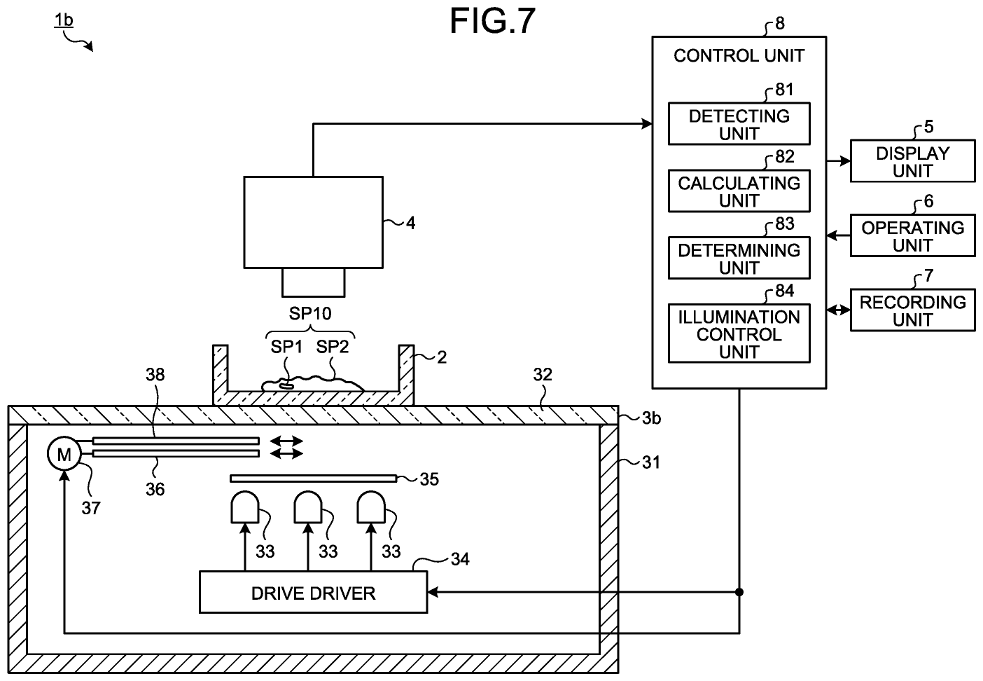

FIG. 7 is a schematic diagram illustrating a schematic configuration of a specimen-sample estimation apparatus according to a third embodiment. A specimen-sample evaluation apparatus 1b illustrated in FIG. 7 includes an illuminating unit 3b in place of the illuminating unit 3a of the specimen-sample evaluation apparatus 1a according to the second embodiment described above.

The illuminating unit 3b irradiates white light and special light that have wavelength bands different from each other to the specimen sample SP10 contained in the container for examination, while switching therebetween, under control of the control unit 8. The illuminating unit 3b further includes a special light filter 38 in addition to the components of the illuminating unit 3a according to the second embodiment described above.

The special light filter 38 transmits light having a wavelength band different from that of the special light filter 36. Specifically, for the special light filter 38, the transmission property is set to transmit the light of the wavelength band, 900 nm to 950 nm, with which the contrast between fat and hemoglobin is high described in the modification of the second embodiment above, and a half width is set to 30 nm or smaller.

In the specimen-sample estimation apparatus 1b thus configured, the illumination control unit 84 drives the driving unit 37 based on a type signal indicating a type of the specimen sample SP10 input from the operating unit 6, and inserts either one out of the special light filter 36 and the special light filter 38 to the optical path of the light emitting unit 33. Specifically, when a type signal indicating that the type of the specimen sample SP10 is collagen tissue is input from the operating unit 6, in the specimen-sample estimation apparatus 1b, the illumination control unit 84 drives the driving unit 37, and inserts the special light filter 36 to the optical path of the light emitting unit 33. On the other hand, when a type signal indicating that the type of the specimen sample SP10 is fat tissue is input from the operating unit 6, in the specimen-sample estimation apparatus 1b, the illumination control unit 84 drives the driving unit 37, and inserts the special light filter 38 to the optical path of the light emitting unit 33. As a result, the specimen-sample evaluation apparatus 1b is enabled to irradiate special light of an appropriate wavelength band to the specimen sample SP10, according to the type of the specimen sample SP10.

According to the third embodiment described above, in the specimen-sample estimation apparatus 1b, while the illumination control unit 84 drives the driving unit 37 to insert the special light filter 36 to the optical path of the light emitting unit 33 when the type signal indicating that the type of the specimen sample SP10 is collagen is input from the operating unit 6, the illumination control unit 84 drives the driving unit 37 to insert the special light filter 38 to the optical path of the light emitting unit 33 when the type signal indicating the type of the specimen sample SP10 is fat tissue is input from the operating unit 6. Therefore, it is possible to irradiate special light of an appropriate wavelength band to the specimen sample SP10, according to the types of the specimen sample SP10.

### Fourth Embodiment

Next, a fourth embodiment will be described. A specimen-sample estimation apparatus according to the fourth embodiment differs in configuration from the specimen-sample estimation apparatus 1 according to the first embodiment described above, and also differ in processing performed thereby. Specifically, while it is determined whether an amount of tissue is equal to or larger than a threshold without separating a living tissue, such as collagen, and blood in the first embodiment described above, it is determined whether an amount of tissue is equal to or larger than a threshold after separating a blood region of blood and a tissue specimen region of a tissue specimen in the fourth embodiment. In the following, after describing a configuration of the specimen-sample estimation apparatus according to the fourth embodiment, processing performed by the specimen-sample estimation apparatus according to the fourth embodiment will be described. Note that identical reference signs are assigned to components identical to those in the specimen-sample estimation apparatus 1 according to the first embodiment, and explanation thereof will be omitted.

Configuration of Specimen-Sample Estimation Apparatus

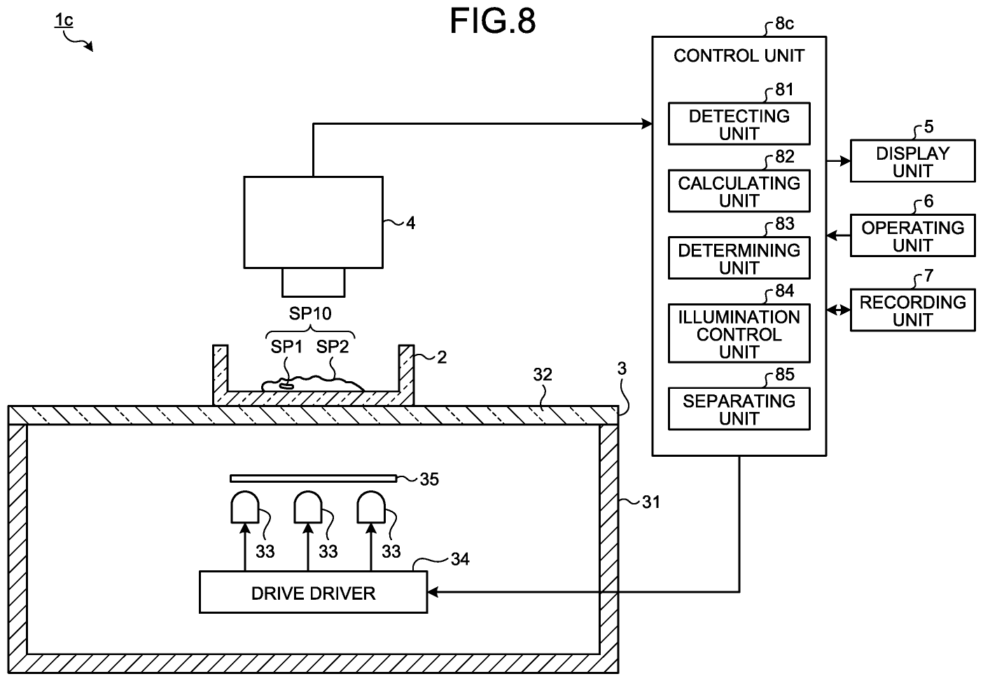

FIG. 8 is a schematic diagram illustrating a schematic configuration of the specimen-sample estimation apparatus according to the fourth embodiment. A specimen-sample estimation apparatus 1c illustrated in FIG. 8 includes a control unit 8c in place of the control unit 8 of the specimen-sample estimation apparatus 1 according to the first embodiment described above.

The control unit 8c includes a separating unit 85 in addition to the control unit 8 according to the first embodiment described above.

The separating unit 85 calculates brightness of a color area of each pixel, and separates a pixel having the brightness equal to or higher than a predetermined threshold as a tissue specimen region, and separates a pixel having the brightness lower than the predetermined threshold as a blood region, for an image corresponding to image data generated by the imaging unit 4.

Processing of Specimen-Sample Estimation Apparatus

Next, processing performed by the specimen-sample estimation apparatus 1c will be described.

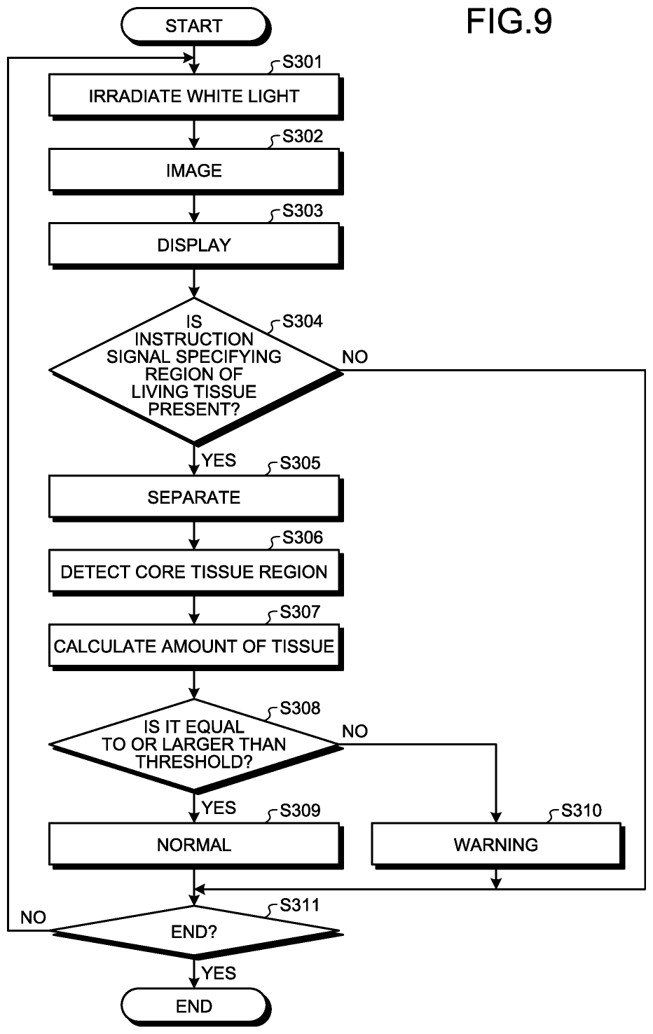

FIG. 9 is a flowchart illustrating an outline of the processing performed by the specimen-sample estimation apparatus 1c. In FIG. 9, step S301 to step S304 correspond to step S101 to step S104 in FIG. 2 described above, respectively.

At step S305, the separating unit 85 calculates brightness of a color area of each pixel, and separates a pixel having the brightness equal to or higher than a predetermined threshold as a tissue specimen region, and separates a pixel having the brightness lower than the predetermined threshold as a blood region for an image corresponding to image data generated by the imaging unit 4. After step S305, the specimen-sample estimation apparatus 1c shifts to step S306.

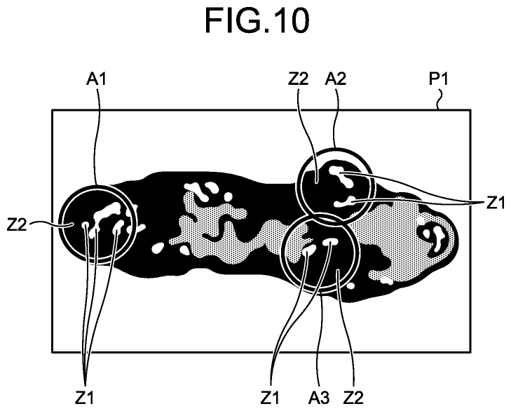

FIG. 10 illustrates an example of an image when the separating unit 85 separates a blood region and a tissue specimen region. As illustrated in FIG. 10, the separating unit 85 calculates brightness of a color region of each pixel for each of regions A1 to A3 specified within an image P1 according to an instruction signal input from the operating unit 6, and separates a pixel having the brightness equal to or higher than a predetermined threshold as a tissue specimen region, and separates a pixel having the brightness lower than the predetermined threshold as a blood region. More specifically, the separating unit 85 separates a region Z1 (region without hatching) in the region A1 as the tissue specimen region, and separates a region Z2 (region in black) as the blood region.

At step S306, the detecting unit 81 detects a region separated by the separating unit 85 as a core tissue region. After step S306, the specimen-sample estimation apparatus 1c shifts to step S307. Step S307 to step S311 correspond to step S106 to step S110 in FIG. 2 described above, respectively.

According to the fourth embodiment described above, it is possible to determine an amount of tissue of a living tissue more accurately.

### Fifth Embodiment

Next, a fifth embodiment will be described. In the fifth embodiment, a region of a living tissue included in a specimen sample is automatically detected by using a learning device that has learned multiple image data groups by machine learning, such as deep learning. In the following, after a configuration of a specimen-sample estimation apparatus according to the fifth embodiment is described, processing performed by the specimen-sample estimation apparatus according to the fifth embodiment will be described. Note that identical reference signs are assigned to components identical to those in the specimen-sample estimation apparatus 1 according to the first embodiment, and explanation thereof will be omitted.

Configuration of Specimen-Sample Estimation Apparatus

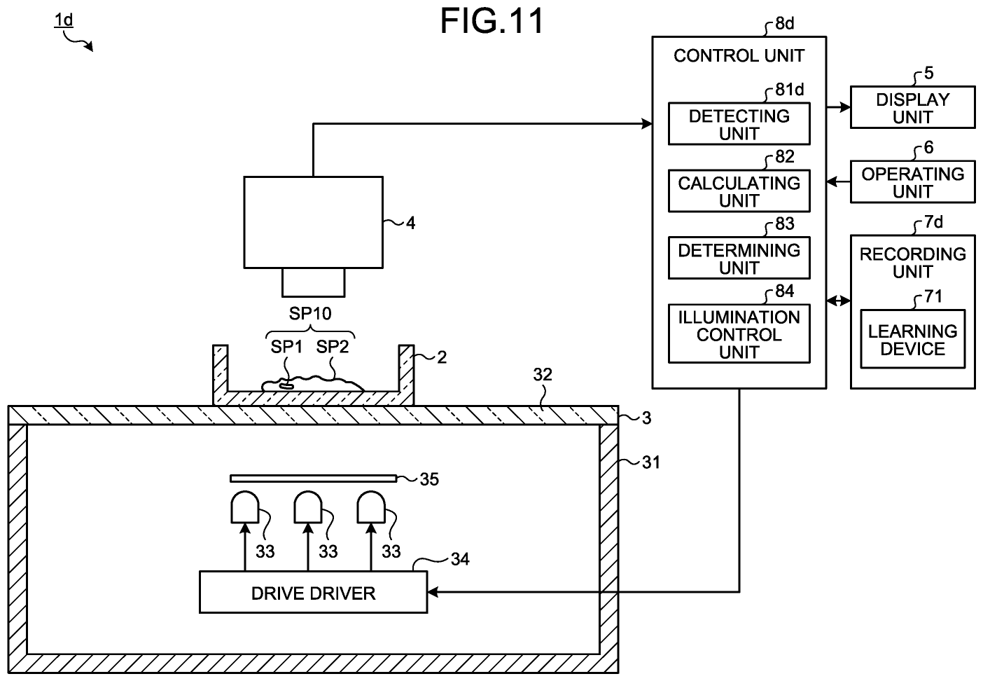

FIG. 11 is a schematic diagram illustrating a schematic configuration of the specimen-sample estimation apparatus according to the fifth embodiment. A specimen-sample estimation apparatus 1d illustrated in FIG. 11 includes a recording unit 7d and a control unit 8d in place of the recording unit 7 and the control unit 8 according to the first embodiment described above.

The recording unit 7d includes a learning device 71 that has learnt feature amounts of a living tissue by learning an image data group generated by imaging plural specimen samples collected by biopsy or puncture needle by machine learning, such as deep learning.

The control unit 8d includes a detecting unit 81d in place of the detecting unit 81 of the control unit 8 according to the first embodiment.

The detecting unit 81d detects a region of a living tissue from an image corresponding to image data generated by the imaging unit 4 by using the learning device 71.

Processing of Specimen-Sample Estimation Apparatus

Next, the processing performed by the specimen-sample estimation apparatus 1d will be described.

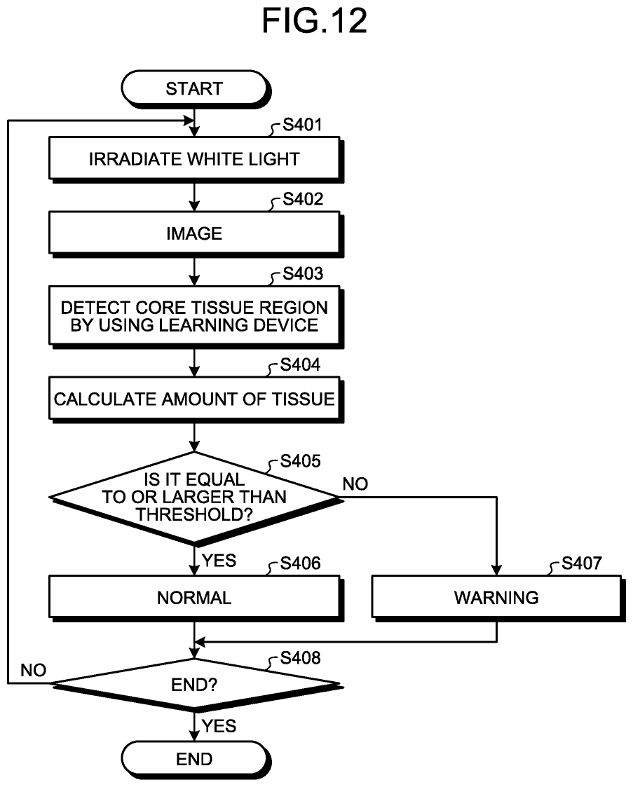

FIG. 12 is a flowchart illustrating an outline of the processing performed by the specimen-sample estimation apparatus 1d. In FIG. 12, step S401 to step S402 correspond to step S101 to step S102 in FIG. 2 described above, respectively.

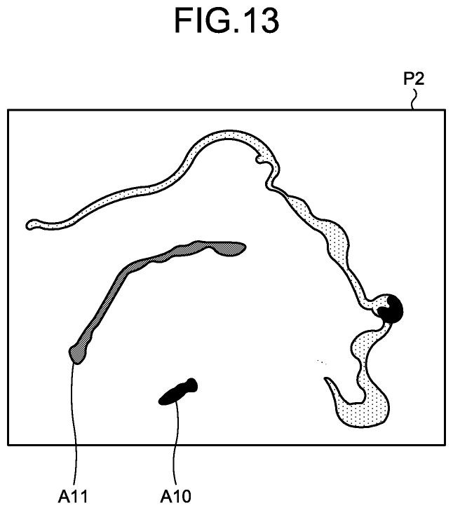

At step S403, the detecting unit 81d detects a core tissue region of a living tissue from an image corresponding to image data generated by the imaging unit 4 by using the learning device 71. Specifically, as illustrated in FIG. 13, by using the learning device 71, the detecting unit 81d detects a long and narrow core tissue region A10 from an image P2, but exclude a long and narrow tissue A11 appearing in the image P2 from the core tissue region. After step S403, the specimen-sample estimation apparatus 1d shifts to step S404 described later. Step S404 to step S408 correspond to step S106 to step S109 in FIG. 2 described above, respectively.

According to the fifth embodiment described above, because a core tissue region is automatically detected, it is possible to reduce a load of an examination operator, such as a doctor.

In the fifth embodiment, the detecting unit 81d detects a core tissue region of a living tissue from an image corresponding to image data generated by the imaging unit 4 by using the learning device 71 provided in the recording unit 7, but it may be configured to detect a core tissue region by using a learning device provided in a server through a network.

### Other Embodiments

By combining components disclosed in the first to the fifth embodiments of the present disclosure described above, various embodiments can be formed. For example, some of the components may be removed from the entire components described in the first to the fifth embodiments of the disclosure described above. Furthermore, the components described in the first to the fifth embodiments of the present disclosure described above may be combined.

Moreover, the control unit and the illuminating unit are separated from each other in the first to the fifth embodiments of the present disclosure, but the control unit and the illuminating unit may be formed in an integrated manner.

Furthermore, in the first to the fifth embodiments of the present disclosure, a term “unit” used in description above may be replaced with “means” or “circuit”. For example, the control unit may be read as control means or control circuit.

Moreover, a computer program to be executed by the specimen-sample estimation apparatus according to the present disclosure is recorded in a computer-readable recording medium, such as a compact disk read-only memory (CD-ROM), a flexible disk (FD), a compact disk recordable (CD-R), a digital versatile disk (DVD), a universal serial bus (USB) medium, and a flash memory, in a file data of a installable format or an executable format, to be provided.

Furthermore, a computer program to be executed by the specimen-sample estimation apparatus according to the present disclosure may be stored in a computer connected to a network, such as the Internet, and be provided by being downloaded through the network. Moreover, a computer program to be executed by the specimen-sample estimation apparatus according to the present disclosure may be provided or distributed through a network, such as the Internet.

Although a sequential relation of processing among steps is specified by using expressions, such as “first”, “thereafter”, and “subsequently”, in the description of the flowcharts in the present specification, it is noted that the order of processing to implement the present disclosure is not uniquely specified by those expressions. That is, the order of processing in the flowcharts described in the present specification may be changed within a range not causing a contradiction. Furthermore, the computer program is not limited to be of simple branch processing as described above. Branching may be performed by generally determining more determination points. In that case, a technique of artificial intelligence that achieves machine learning while prompting a user for manual operations and repeating learning may be used in combination. Moreover, it may be configured to learn operating patterns performed by many specialists, and to perform deep learning by applying further complicated conditions.

According to the present disclosure, it is possible to estimate whether a sufficient amount of tissue of a living tissue necessary for pathological diagnosis is obtained in a specimen sample tissue without depending on subjectivity of an estimator.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A specimen-sample estimation apparatus comprising:
a light emitter configured to emit white light to irradiate light to a specimen sample including a living tissue;
a plurality of special light filters that are insertable and removable to and from an optical path of the white light emitted by the light emitter, each special light filter being configured to transmit light having a different wavelength band;
a driver configured to move one of the special light filters to the optical path;
an imager configured to image the specimen sample to which the light has been irradiated by the light emitter, to generate image data; and
a processor comprising hardware, the processor being configured to
detect a core tissue region of the living tissue appearing in an image corresponding to the image data generated by the imager,
calculate an amount of tissue of the living tissue, based on the detected core tissue region, and
determine whether the calculated amount of tissue is equal to or larger than a predetermined threshold.

2. The specimen-sample estimation apparatus according to claim 1, wherein
the processor is configured to calculate at least one of length, width, area, and volume of the core tissue region, as the amount of tissue.

3. The specimen-sample estimation apparatus according to claim 1, further comprising
an operating circuit configured to receive an input of an instruction signal specifying a region of the living tissue appearing in the image, wherein
the processor is configured to detect the core tissue region based on the received instruction signal.

4. The specimen-sample estimation apparatus according to claim 1, wherein
the processor is further configured to drive the driver to move the special light filter according to a type of the specimen sample to the optical path.

5. The specimen-sample estimation apparatus according to claim 1, wherein
A peak band of the wavelength band is within a range of 400 nanometers (nm) to 450 nm, a range of 580 nm to 650 nm, or a range of 900 nm to 950 nm, and a half width of the wavelength band is set to 30 nm or smaller.

6. The specimen-sample estimation apparatus according to claim 1, wherein
the specimen sample is collected by either one of a puncture needle, forceps, and a brush.

7. The specimen-sample estimation apparatus according to claim 6, wherein
the processor is configured to calculate the amount of tissue based on a marker indicating a dimension engraved or printed on a specimen sample container.

8. The specimen-sample estimation apparatus according to claim 6, wherein
the processor is configured to calculate the amount of tissue based on a diameter of the puncture needle.

9. The specimen-sample estimation apparatus according to claim 1, further comprising
an output circuit configured to output a result of determination by the processor.

10. A specimen-sample estimation method comprising:
moving one of a plurality of special light filters to an optical path of white light, each special light filter being configured to transmit light having a different wavelength band;
irradiating the white light to a specimen sample including a living tissue;
imaging the specimen sample to which the white light has been irradiated, to generate image data;
detecting a core tissue region of the living tissue appearing in an image corresponding to the image data;
calculating an amount of tissue of the living tissue based on the core tissue region; and
determining whether the amount of tissue is equal to or larger than a predetermined threshold.

11. A non-transitory computer-readable recording medium with an executable program stored thereon, the program causing a specimen-sample estimation apparatus to execute:
moving one of a plurality of special light filters to an optical path of white light, each special light filter being configured to transmit light having a different wavelength band;
irradiating the white light to a specimen sample including a living tissue;
imaging the specimen sample to which the white light has been irradiated, to generate image data;
detecting a core tissue region of the living tissue appearing in an image corresponding to the image data;
calculating an amount of tissue of the living tissue based on the core tissue region; and
determining whether the amount of tissue is equal to or larger than a predetermined threshold.
